(19) United States
(12) Patent Application Publication
(10) Pub. No.: US 2020/0289574 A1
(43) Pub. Date: Sep. 17, 2020

(54) AUGMENTATION OF FERTILITY BY PLATELET RICH PLASMA

(71) Applicant: ARTERIOCYTE MEDICAL SYSTEMS, INC., St. Louis, MO (US)

(72) Inventors: Donald BROWN, St. Louis, MO (US); Lusine AGHAJANOVA, St. Louis, MO (US)

(21) Appl. No.: 16/759,671

(22) PCT Filed: Oct. 29, 2018
(86) PCT No.: PCT/US18/58012

§ 371 Date: Apr. 27, 2020

Related U.S. Application Data
(60) Provisional application No. 62/577,727, filed on Oct. 27, 2017.

(51) Int. Cl. A61K 35/19; A61P 15/08; A61K 35/28; A61K 35/16; A61K 38/18; A61K 38/30; A61K 35/35; A61K 38/19

(52) U.S. Cl. CPC A61K 35/19; A61P 15/08; A61K 35/28; A61K 35/16; A61K 2035/124; A61K 38/30; A61K 38/1866; A61K 35/35; A61K 38/193; A61K 38/1808

(57) ABSTRACT

Methods and compositions for stimulating ovarian function in a patient suffering from premature ovarian failure and for decreasing recurrent spontaneous abortion are disclosed.

### CROSS REFERENCES TO RELATED APPLICATIONS

This application claims the priority of provisional Application No. 62/577,727, filed Oct. 27, 2017, the entire disclosure of which is incorporated herein by reference.

### FIELD OF THE INVENTION

Disclosed are treatments, protocols and compositions of matter useful for enhancing female fertility comprising administration of platelet rich plasma, and/or platelet lysate into fertility associated tissues in a subject in need of therapy.

### BACKGROUND OF THE INVENTION

The belief that all primary follicles in adult mammalian females were formed during the fetal period has persisted for over fifty years, primarily due to the diminution in the number of primary follicles that occurs with age. Recent studies, however, have brought this belief into question. For example, Bukovsky et al. (Am J Reprod Immunol, 1995. 33(4): p. 323-40) reported that the ovarian surface epithelium (OSE) is a source of germ cells in adult human females. It was also reported that new primary follicles are formed by assembly of oocytes with nests of primitive granulosa cells in the human ovarian cortex during adulthood (Reprod Biol Endocrinol, 2004. 2: p. 20). These data suggest that the hypothesis that all primary follicles in adult human females were formed during the fetal period is incorrect. Rather, these studies indicate that primary follicles that were formed during the fetal period may persist for some time, perhaps through childhood, and then starting with menarche, new cohorts of primary follicles replace the fetal follicles that undergo atresia. Moreover, it is suggested that during each successive menstrual cycle until about the age of 38, fresh follicles are produced that replace older follicles. At about the age of 38+/−2 years, formation of new primary follicles ceases, possibly due to the onset of immune senescence (certain immune system-related cells appear to be required for the formation of new germ cells from the OSE), and the aging follicles accumulate genetic alterations until exhausted at the onset of natural menopause.

It is known that women in developed countries have an increased tendency to delay having a first child. Reasons for waiting to start a family include the desire to wait until the family has financial security and the desire of women to commit to their marriages and to their careers before focusing their energy on children. Specifically, the National Center for Health Statistics reports that the birth rate for women aged 40-45 years rose 20% between 1990 and 1995, and increased 74% during 1981-95. The rising birth rate along with the increasing number of women in this age group means that there were more babies born in 1995 to mothers in their 40s than in any year since 1966.

A significant problem that occurs due to delayed parenting is that often women over the age of 40 have a greatly decreased chance of becoming pregnant. Much of this difficulty stems from a decrease in the availability of oocytes that are available for fertilization. Additionally, women below the age of 40 that have premature ovarian failure are unable to become pregnant due to the lack of production of oocytes. Other factors include inflammation processes, and/or accelerated deterioration of the follicular environment that causes loss of oocyte viability and function.

At present, a woman who desires to become pregnant but who does not produce her own oocytes may elect to undergo a procedure referred to as in vitro fertilization (IVF) in which eggs donated from another woman are fertilized in vitro and then one or two of the resultant developing embryos are implanted into her uterus. Of course, the resultant child from this procedure will be genetically unrelated to the woman undergoing this procedure. At the present time, there are no procedures available by which a woman who does not produce oocytes is able to produce a child who is genetically related to her.

Therefore, there is a need for means of enhancing oocyte maturation, and stimulating processes of regeneration in order to allow for enhanced fertility.

### SUMMARY OF THE INVENTION

One aspect of the present disclosure encompasses a method of stimulating ovarian function in a patient suffering from premature ovarian failure. The method comprises the steps of: a) obtaining peripheral blood; b) isolating platelet rich plasma, and/or platelet lysate; c) quantifying growth factor content of said platelet rich plasma and/or platelet lysate; d) optionally concentrating said growth factors from said platelet rich plasma and/or platelet lysate derived growth factors; and e) administering said growth factors locally into said ovarian tissue in a patient in need of treatment.

Administration into ovarian tissue may be performed at a concentration and frequency sufficient to induce differentiation of oocyte progenitor cells. Alternatively, administration into ovarian tissue may be performed at a concentration and frequency sufficient to induce reduction of fibrotic damage to said ovarian tissue. Administration into ovarian tissue may also be performed at a concentration and frequency sufficient to induce reduction of IL-17 in said ovarian tissue.

Another aspect of the present disclosure encompasses a method of decreasing recurrent spontaneous abortion. The method comprises: a) extracting an autologous population of regenerative cells; b) treating said autologous population of regenerative cells with platelet rich plasma, and/or platelet lysate at a concentration and duration sufficient to induce type 2 cytokine polarization; and c) administering said treated regenerative cells into a patient in need of treatment.

Autologous regenerative cells may be bone marrow mononuclear cells. Alternatively, autologous regenerative cells may be adipose stromal vascular fraction cells. Autologous regenerative cells may also be peripheral blood mononuclear cells.

When autologous regenerative cells are peripheral blood mononuclear cells, the cells may be collected subsequent to administration of a mobilization means. Mobilization means may be G-CSF, Mozibil, FLR-3L, or combinations thereof.

Type 2 cytokine polarization may be associated with reduction in ability to produce interferon gamma after mitogenic stimulation. Mitogenic stimulation may be treatment with phytohemagluttinin at a concentration of 2 micrograms per ml. Type 2 cytokine polarization may also be associated with enhanced IL-4 production after mitogenic stimulation. Mitogenic stimulation may be treatment with phytohemagluttinin at a concentration of 2 micrograms per ml.

### DETAILED DESCRIPTION

Certain embodiments commensurate in scope with the originally claimed invention are summarized below. These embodiments are not intended to limit the scope of the claimed invention, but rather these embodiments are intended only to provide a brief summary of possible forms of the invention. Indeed, the invention may encompass a variety of forms that may be similar to or different from the embodiments set forth below. One or more specific embodiments of the present subject matter will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The term “platelet plasma” as used herein refers to platelet rich plasma (PRP), human platelet lysate (HPL), and combinations thereof.

“Platelet rich plasma” (PRP) as described herein is a blood plasma that has been enriched with platelets. As a concentrated source of autologous platelets, PRP contains and releases several different growth factors and other cytokines that stimulate healing of bone and soft tissue. Components of PRP may include but are not limited to platelet-derived growth factor, transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, and combinations thereof.

PRP may be prepared by collection of the patient's whole blood (that is anticoagulated with citrate dextrose) before undergoing two stages of centrifugation designed to separate the PRP aliquot from platelet-poor plasma and red blood cells. In humans, a typical baseline blood platelet count may range from about 150,000 to about 450,000 platelets per μl of blood, or about 200,000 platelets per μl of blood. Therapeutic PRP may concentrate platelets in plasma by about five-fold. As such, PRP platelet count in PRP may range from about 750,000 to about 2.25×10⁶ platelets per μl of PRP, or about 1×10⁶ platelets per μl of blood. The PRP may then be used to prepare human platelet lysate.

Compositions of the present disclosure may comprise platelet plasma compositions from PRP, HPL, or combinations thereof, and either platelet plasma composition may be used to regenerate ovarian tissue for augmentation of fertility. Further, the platelet plasma composition may be used with or without concentrated bone marrow (BMAC). By way of example, when administered into ovarian tissue, about 0.05 to about 2.0 cc of platelet plasma composition may be used. Platelets are non-nucleated blood cells that as noted above are found in bone marrow and peripheral blood.

In various embodiments of the present invention, the platelet plasma composition may be obtained by sequestering platelets from whole blood or bone marrow through centrifugation, for example into three strata: (1) platelet rich plasma; (2) platelet poor plasma; and (3) fibrinogen. When using platelets from one of the strata, e.g., the platelet rich plasma (PRP) from blood, one may use the platelets whole or their contents may be extracted and concentrated into a platelet lysate through a cell membrane lysis procedure using thrombin and/or calcium chloride, for example. When choosing whether to use the platelets whole or as a lysate, one may consider the rate at which one desires ovarian tissue regeneration. In some embodiments the lysate will act more rapidly than the PRP (or platelet poor plasma from bone marrow).

Human platelet lysate may be formed from but not limited to PRP, pooled platelets from humans, and cultured megakaryocytes from stem cell expansion technology. In some embodiments, HPL is from a commercial source. In some embodiments, HPL is prepared in the laboratory from platelet rich plasma (PRP), pooled platelets from humans, or cultured megakaryocytes from stem cell expansion technology.

Notably, platelet poor plasma that is derived from bone marrow has a greater platelet concentration than platelet rich plasma from blood, also known as platelet poor/rich plasma (“PP/RP” or “PPP”). PP/RP or PPP may be used to refer to platelet poor plasma derived from bone marrow, and in some embodiments, preferably PP/RP is used or PRP is used as part of the composition for disc regeneration. (By convention, the abbreviation PRP refers only to compositions derived from peripheral blood and PPP (or PP/RP) refers to compositions derived from bone marrow.) In various embodiments, the platelet plasma composition, which may or may not be in the form of a lysate, may serve one or more of the following functions: (1) to release/provide growth factors and cytokines for tissue regeneration; (2) to reduce inflammation; (3) to attract/mobilize cell signaling; (4) to initiate repair of damaged/atrophied ovarian tissue through fibroblast growth factors (FGF); (5) to stabilize extracellular matrix in the ovary; (6) to stimulate maturation of immature oocytes; (7) to stimulate revascularization of fibrotic tissue; and (8) to stimulate oocyte receptivity to spermatozoa. Additionally, by combining platelet therapy with stem cells, there can be synergy with respect to augmentation of fertility.

In some embodiments in which the lysate is used, the cytokines may be concentrated in order to optimize their functional capacity. Concentration may be accomplished in two steps. First, blood may be obtained and concentrated to a volume that is 5-15% of what it was before concentration. Devices that may be used include but are not limited to a hemofilter or a hemo-concentrator. For example, 60 cc of blood may be concentrated down to 6 cc. Next, the concentrated blood may be filtered to remove water. This filtering step may reduce the volume further to 33%-67% (e.g., approximately 50%) of what it was prior to filtration. Thus, by way of example for a concentration product of 6 cc, one may filter out water so that one obtains a product of approximately 3 cc. When the platelet rich plasma, platelet poor plasma and fibrinogen are obtained from blood, they may for example be obtained by drawing 20-500 cc of peripheral blood, 40-250 cc of peripheral blood, or 60-100 cc of peripheral blood. The amount of blood that one should draw will depend on the extent of ovarian tissue degeneration.

In some embodiments, a method of generation of said PRP may be used according to U.S. Pat. No. 9,011,929, which is incorporated by reference herein in its entirety. In essence, a method may comprise separating PRP from whole blood by collecting whole blood from an animal or patient into a vacuum test tube containing sodium citrate, and primarily centrifuging the collected whole blood; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid; and collecting the PRP concentrated in a bottom layer by another blunt needle; mixing the PRP collected from the separating step with a calcium chloride solution by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at room temperature before mixing; and mixing the mixture of the PRP and the calcium chloride solution with the type I collagen, in an opaque phase, four times by another three-way connector.

In an exemplary embodiment of the disclosure, a method may comprise separating the PRP from whole blood, wherein the separating step further comprises the steps of: collecting 10 ml of the whole blood from an animal or patient into a vacuum test tube containing 3.2% sodium citrate, and primarily centrifuging the collected whole blood at 1,750-1,900 g for 3 to 5 minutes; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid at 4,500-5,000 g for 4 to 6 minutes; and collecting the PRP concentrated in a bottom layer by another blunt needle; mixing 1 mL of the PRP collected from the separating step with a calcium chloride solution with a concentration of 0.30-0.55 mg/mL by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at a room temperature for 15 to 30 minutes before mixing; and mixing the mixture of the PRP and the calcium chloride solution with the type I collagen with a concentration of 20-50 mg/mL, in an opaque phase, four times by another three-way connector.

The term “platelet-rich plasma” or “PRP” as used herein is a broad term which is used in its ordinary sense and is a concentration of platelets greater than the peripheral blood concentration suspended in a solution of plasma, or other excipient suitable for administration to a human or non-human animal including, but not limited to, isotonic sodium chloride solution, physiological saline, normal saline, dextrose 5% in water, dextrose 10% in water, Ringer solution, lactated Ringer solution, Ringer lactate, Ringer lactate solution, and the like. PRP compositions may be an autologous preparation from whole blood taken from the subject to be treated or, alternatively, PRP compositions may be prepared from a whole blood sample taken from a single donor source or from whole blood samples taken from multiple donor sources. In general, PRP compositions comprise platelets at a platelet concentration that is higher than the baseline concentration of the platelets in whole blood. In some embodiments, PRP compositions may further comprise WBCs at a WBC concentration that is higher than the baseline concentration of the WBCs in whole blood. As used herein, baseline concentration means the concentration of the specified cell type found in the patient's blood which would be the same as the concentration of that cell type found in a blood sample from that patient without manipulation of the sample by laboratory techniques such as cell sorting, centrifugation or filtration. Where blood samples are obtained from more than one source, baseline concentration means the concentration found in the mixed blood sample from which the PRP is derived without manipulation of the mixed sample by laboratory techniques such as cell sorting, centrifugation or filtration. In some embodiments, PRP compositions comprise elevated concentrations of platelets and WBCs and lower levels of RBCs and hemoglobin relative to their baseline concentrations. In some embodiments of PRP composition, only the concentration of platelets is elevated relative to the baseline concentration. Some embodiments of PRP composition comprise elevated levels of platelets and WBCs compared to baseline concentrations. In some embodiments, PRP compositions comprise elevated concentrations of platelets and lower levels of neutrophils relative to their baseline concentrations. Some embodiments of PRP composition comprise elevated levels of platelets and neutrophil-depleted WBCs compared to their baseline concentrations. In some embodiments of PRP, the ratio of lymphocytes and monocytes to neutrophils is significantly higher than the ratios of their baseline concentrations. The PRP formulation may include platelets at a level of between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, about 11 and about 12 times the baseline, about 12 and about 13 times the baseline, about 13 and about 14 times the baseline, or higher. In some embodiments, the platelet concentration may be between about 4 and about 6 times the baseline. Typically, a microliter of whole blood comprises at least 140,000 to 150,000 platelets and up to 400,000 to 500,000 platelets. The PRP compositions may comprise about 500,000 to about 7,000,000 platelets per microliter. In some instances, the PRP compositions may comprise about 500,000 to about 700,000, about 700,000 to about 900,000, about 900,000 to about 1,000,000, about 1,000,000 to about 1,250,000, about 1,250,000 to about 1,500,000, about 1,500,000 to about 2,500,000, about 2,500,000 to about 5,000,000, or about 5,000,000 to about 7,000,000 platelets per microliter. The WBC concentration is typically elevated in PRP compositions. For example, the WBC concentration may be between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, or higher. The WBC count in a microliter of whole blood is typically at least 4,100 to 4,500 and up to 10,900 to 11,000. The WBC count in a microliter of the PRP composition may be between about 8,000 and about 10,000; about 10,000 and about 15,000; about 15,000 and about 20,000; about 20,000 and about 30,000; about 30,000 and about 50,000; about 50,000 and about 75,000; and about 75,000 and about 100,000. Among the WBCs in the PRP composition, the concentrations may vary by the cell type but, generally, each may be elevated. In some variations, the PRP composition may comprise specific concentrations of various types of white blood cells. The relative concentrations of one cell type to another cell type in a PRP composition may be the same or different than the relative concentration of the cell types in whole blood. For example, the concentrations of lymphocytes and/or monocytes may be between about 1.1 and about 2 times baseline, about 2 and about 4 times baseline, about 4 and about 6 times baseline, about 6 and about 8 times baseline, or higher. In some variations, the concentrations of the lymphocytes and/or the monocytes may be less than the baseline concentration. The concentrations of eosinophils in the PRP composition may be less than baseline, about 1.5 times baseline, about 2 times baseline, about 3 times baseline, about 5 times baseline, or higher.

In whole blood, the lymphocyte count is typically between 1,300 and 4,000 cells per microliter, but in other examples, the lymphocyte concentration may be between about 5,000 and about 20,000 per microliter. In some instances, the lymphocyte concentration may be less than 5,000 per microliter or greater than 20,000 per microliter. The monocyte count in a microliter of whole blood is typically between 200 and 800. In the PRP composition, the monocyte concentration may be less than about 1,000 per microliter, between about 1,000 and about 5,000 per microliter, or greater than about 5,000 per microliter. The eosinophil concentration may be between about 200 and about 1,000 per microliter elevated from about 40 to 400 in whole blood. In some variations, the eosinophil concentration may be less than about 200 per microliter or greater than about 1,000 per microliter.

In certain variations, the PRP composition may contain a specific concentration of neutrophils. The neutrophil concentration may vary between less than the baseline concentration of neutrophils to eight times than the baseline concentration of neutrophils. In some embodiments, the PRP composition may include neutrophils at a concentration of 50-70%, 30-50%, 10-30%, 5-10%, 1-5%, 0.5-1%, 0.1-0.5% of levels of neutrophils found in whole blood or even less. In some embodiments, neutrophil levels are depleted to 1% or less than that found in whole blood. In some variations, the neutrophil concentration may be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration may additionally or alternatively be specified relative to the concentration of the lymphocytes and/or the monocytes. One microliter of whole blood typically comprises 2,000 to 7,500 neutrophils. In some variations, the PRP composition may comprise neutrophils at a concentration of less than about 1,000 per microliter, about 1,000 to about 5,000 per microliter, about 5,000 to about 20,000 per microliter, about 20,000 to about 40,000 per microliter, or about 40,000 to about 60,000 per microliter. In some embodiments, neutrophils are eliminated or substantially eliminated. Means to deplete blood products, such as PRP, of neutrophils is known and discussed in U.S. Pat. No. 7,462,268, which is incorporated herein by reference. Several embodiments are directed to PRP compositions in which levels of platelets and white blood cells are elevated compared to whole blood and in which the ratio of monocytes and/or lymphocytes to neutrophils is higher than in whole blood. The ratio of monocytes and/or lymphocytes to neutrophils may serve as an index to determine if a PRP formulation may be efficaciously used as a treatment for a particular disease or condition. PRP compositions where the ratio of monocytes and/or lymphocytes to neutrophils is increased may be generated by either lowering neutrophils levels, or by maintaining neutrophil levels while increasing levels of monocytes and/or lymphocytes. Several embodiments relate to a PRP formulation that contains 1.01 times, or higher, baseline platelets in combination with a 1.01 times, or higher, baseline white blood cells with the neutrophil component depleted at least 1% from baseline. In some embodiments, the PRP compositions may comprise a lower concentration of red blood cells (RBCs) and/or hemoglobin than the concentration in whole blood. The RBC concentration may be between about 0.01 and about 0.1 times baseline, about 0.1 and about 0.25 times baseline, about 0.25 and about 0.5 times baseline, or about 0.5 and about 0.9 times baseline. The hemoglobin concentration may be depressed and in some variations may be about 1 g/dl or less, between about 1 g/dl and about 5 g/dl, about 5 g/dl and about 10 g/dl, about 10 g/dl and about 15 g/dl, or about 15 g/dl and about 20 g/dl. Typically, whole blood drawn from a male patient may have an RBC count of at least 4,300,000 to 4,500,000 and up to 5,900,000 to 6,200,000 per microliter while whole blood from a female patient may have an RBC count of at least 3,500,000 to 3,800,000 and up to 5,500,000 to 5,800,000 per microliter. These RBC counts generally correspond to hemoglobin levels of at least 132 g/L to 135 g/L and up to 162 g/L to 175 g/L for men and at least 115 g/L to 120 g/L and up to 152 g/L to 160 g/L for women. In some embodiments, PRP compositions contain increased concentrations of growth factors and other cytokines. In several embodiments, PRP compositions may include increased concentrations of one or more of: platelet-derived growth factor, transforming growth factor beta, fibroblast growth factor, insulin-like growth factor, insulin-like growth factor 2, vascular endothelial growth factor, epidermal growth factor, interleukin-8, keratinocyte growth factor, and connective tissue growth factor. In some embodiments, the platelets collected in PRP are activated by thrombin and calcium chloride to induce the release of these growth factors from alpha granules. In some embodiments, a PRP composition is activated exogenously with thrombin and/or calcium to produce a gel that can be applied to an area to be treated. The process of exogenous activation, however, results in immediate release of growth factors. Other embodiments relate to activation of PRP via in vivo contact with collagen containing tissue at the treatment site. The in vivo activation of PRP results in slower growth factor release at the desired site.

In certain embodiments of the invention, the PRP composition may comprise a PRP derived from a human or animal source of whole blood. The PRP may be prepared from an autologous source, an allogenic source, a single source, or a pooled source of platelets and/or plasma. To derive the PRP, whole blood may be collected, for example, using a blood collection syringe. The amount of blood collected may depend on a number of factors, including, for example, the amount of PRP desired, the health of the patient, the severity or location of the tissue damage and/or the MI, the availability of prepared PRP, or any suitable combination of factors. Any suitable amount of blood may be collected. For example, about 1 cc to about 150 cc of blood or more may be drawn. More specifically, about 27 cc to about 110 cc or about 27 cc to about 55 cc of blood may be withdrawn. In some embodiments, the blood may be collected from a patient who may be presently suffering, or who has previously suffered from, connective tissue damage and/or an MI. PRP made from a patient's own blood may significantly reduce the risk of adverse reactions or infection.

In an exemplary embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area. The PRP may be prepared in any suitable way. For example, the PRP may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume.

In some examples, the blood may then be centrifuged using a gravitational platelet system, such as the Cell Factor Technologies GPS System® centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood may be collected from a patient, about 5 cc of PRP may be obtained.

As the PRP composition comprises activated platelets, active agents within the platelets are released. These agents include, but are not limited to, cytokines (e.g., IL-1B, IL-6, TNF-A), chemokines (e.g., ENA-78 (CXCL8), IL-8 (CXCL8), MCP-3 (CCL7), MIP-1A (CCL3), NAP-2 (CXCL7), PF4 (CXCL4), RANTES (CCL5)), inflammatory mediators (e.g., PGE2), and growth factors (e.g., Angiopoitin-1, bFGF, EGF, FGF, HGF, IGF-I, IGF-II, PDAF, PDEGF, PDGF AA and BB, TGF-.beta. 1, 2, and 3, and VEGF).

Said PRP may be used to treat autologous regenerative cells prior to administration of said cells for stimulation of ovary regeneration and/or prevention of immunologically mediated abortions. One type of autologous regenerative cells are adipose stromal vascular fraction cells. Said stromal vascular fraction cells are obtained by the following steps; a) Using aseptic technique and with local anesthesia, the infraumbilical region is infiltrated with 0.5% Xylocaine with 1:200,000 epinephrine; b) After allowing 10 minutes for hemostasis, a 4 mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7 mL/500 mL) is added to the washed lipoaspirate using a 1:1 volume ratio; f) The centrifuge containers are sealed and placed in a 37° C. shaking water bath for one hour then centrifuged for 5 min at 300 rcf; g) Following centrifugation, the stromal cells are resuspended within Isolyte in separate sterile 50 mL centrifuge tubes; h) The tubes are centrifuged for 5 min. at 300 rcf and the Isolyte is removed, leaving cell pellet; i) The pellets are resuspended in 40 ml of Isolyte, centrifuged again for 5 min at 300rcf. The supernatant is again be removed; j) The cell pellets are combined and filtered through 100 □m cell strainers into a sterile 50 ml centrifuge tube and centrifuged for 5 min at 300rcf and the supernatant removed, leaving the pelleted adipose stromal cells. Means of combining PRP and SVF are known in the literature and incorporated by reference [3-7].

In some embodiments, the neutrophils are depleted by at least 5%, in some embodiments, the neutrophils are depleted by at least 10%, in some embodiments, the neutrophils are depleted by at least 15%, in some embodiments, the neutrophils are depleted by at least 20%, in some embodiments, the neutrophils are depleted by at least 25%, in some embodiments, the neutrophils are depleted by at least 30%, in some embodiments, the neutrophils are depleted by at least 35%, in some embodiments, the neutrophils are depleted by at least 40%, in some embodiments, the neutrophils are depleted by at least 45%, in some embodiments, the neutrophils are depleted by at least 50%, in some embodiments, the neutrophils are depleted by at least 55%, in some embodiments, the neutrophils are depleted by at least 60%, in some embodiments, the neutrophils are depleted by at least 65%, in some embodiments, the neutrophils are depleted by at least 70%, in some embodiments, the neutrophils are depleted by at least 75%, in some embodiments, the neutrophils are depleted by at least 80%, in some embodiments, the neutrophils are depleted by at least 85%, in some embodiments, the neutrophils are depleted by at least 90%, in some embodiments, the neutrophils are depleted by at least 95%, in some embodiments, the neutrophils are depleted by at least 95%. In some embodiments, the neutrophils in the platelet rich plasma are substantially removed.

Administration of PRP intraovarially may be performed using methods known in the art. Exemplary publications, which are incorporated by reference for guidance in the practice of the invention are provided [8, 9].

In some embodiments of the invention, autologous regenerative cells such as adipose stromal vascular fraction cells, and/or bone marrow mononuclear cells are administered together with platelet rich plasma and/or platelet lysate.

In one embodiment of the invention, inflammatory and immunological abnormalities are identified in order to categorize risk of pregnancy complications, said pregnancy complications are defined as medical incidences that threaten the health of the mother or the offspring, and include RESA, preterm birth, pre-eclampsia including hemolysis elevated liver enzymes low platelets (HELP), premature rupture of the membrane, Antepartum hemorrhage including placental abruption, chorioamnionitis, Intrauterine growth restriction, placenta pravaevia, sequalae of intraamniotic infection. In one particular embodiment, levels of circulating factors are assessed in maternal plasma, based on abnormally high levels, interventions are chosen for treatment. In one particular embodiment, the methodology of Ruiz et al [10], is utilized for assessment of circulating IL-6. Specifically, plasma is analyzed in the second trimester of pregnancy and concentrations correlated with a baseline associated with non-complicated pregnancy. Within the context of the invention, other markers of inflammation may be utilized such as C reactive protein [11], In females who have higher concentration of inflammatory proteins as compared to baseline values from non-complicated pregnancies, an agent is administered to reduce inflammation. Numerous studies have demonstrated that recurrent spontaneous abortions (RSA) is associated with increased production of Th1 cytokines such as interferon gamma and reduced production of IL-10 [12, 13]. Furthermore, treatments that have demonstrated some signal of efficacy in RSA such as IVIG [14], G-CSF [15], and PLT [16], all have been shown to induce a Th1 to Th2 shift. Within the context of the current invention, use of stem cell mixtures, particularly adipose SVF for inducing immune modulation towards protecting the fetal allograft are envisioned. In one specific embodiment, autologous bone marrow and/or SVF are used to extract autologous Treg, which are activated by PRP treatment and administered into a mammal suffering from RSA at a concentration sufficient to evoke a therapeutic response. Such concentrations may be determined by monitoring NK activity, assessing inflammatory cytokine production by peripheral blood mononuclear cells after stimulation with a mitogen or mitogenic antibody, or by assessment of T regulatory (Treg) cell numbers or activity. In one embodiment RSA patients are administered PRP treated populations of cells that contain CD4+ CD25+ cells at a concentration of 50 million cells, once per month.

### EXAMPLES

The publications discussed above are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Stimulating Ovarian Function in a Patient Suffering from Premature Ovarian Failure

A subject suffering from premature ovarian failure may be treated using a method of the disclosure. First, peripheral blood may be obtained, and platelet rich plasma, and/or platelet lysate isolated from the peripheral blood. Growth factor content of the platelet rich plasma and/or platelet lysate may be quantified. Growth factors may further be concentrated in the platelet rich plasma and/or platelet lysate. The prepared platelet rich plasma and/or platelet lysate may be administered locally into ovarian tissue in a patient in need of treatment.

### Example 2. Decreasing Recurrent Spontaneous Abortion in a Patient

A subject suffering from recurrent spontaneous abortion may be treated using a method of the disclosure. First, an autologous population of regenerative cells may be isolated. The autologous population of regenerative cells may be treated with platelet rich plasma and/or platelet lysate at a concentration and duration sufficient to induce type 2 cytokine polarization. The resulting regenerative cells may be administered into a patient in need of treatment.

1. A method of stimulating ovarian function in a patient suffering from premature ovarian failure comprising the steps of: a) obtaining peripheral blood;
b) isolating platelet rich plasma, and/or platelet lysate; c) quantifying growth factor content of said platelet rich plasma and/or platelet lysate; d) optionally concentrating said growth factors from said platelet rich plasma and/or platelet lysate derived growth factors; and e) administering said growth factors locally into ovarian tissue in a patient in need of treatment.

2. The method of claim 1, wherein said growth factors associated with platelet rich plasma and/or platelet lysate are selected from EGF, IGF, VEGF, PDGF, activin A and combinations thereof.

3. The method of claim 1, wherein said growth factors are exosomes.

4. The method of claim 1, wherein said growth factors are microvesicles.

5. The method of claim 1, wherein said growth factors are subcellular fragments.

6. The method of claim 1, wherein said administration into ovarian tissue is performed at a concentration and frequency sufficient to induce differentiation of oocyte progenitor cells.

7. The method of claim 1, wherein said administration into ovarian tissue is performed at a concentration and frequency sufficient to induce reduction of fibrotic damage to said ovarian tissue.

8. The method of claim 1, wherein said administration into ovarian tissue is performed at a concentration and frequency sufficient to induce reduction of IL-17 in said ovarian tissue.

9. A method of decreasing recurrent spontaneous abortion comprising of: a) extracting an autologous population of regenerative cells; b) treating said autologous population of regenerative cells with platelet rich plasma, and/or platelet lysate at a concentration and duration sufficient to induce type 2 cytokine polarization; and c) administering said treated regenerative cells into a patient in need of treatment.

10. The method of claim 9, wherein said autologous regenerative cells are bone marrow mononuclear cells.

11. The method of claim 9, wherein said autologous regenerative cells are adipose stromal vascular fraction cells.

12. The method of claim 9, wherein said autologous regenerative cells are peripheral blood mononuclear cells.

13. The method of claim 12, wherein said peripheral blood mononuclear cells are collected subsequent to administration of a mobilization means.

14. The method of claim 13, wherein said mobilization means is G-CSF administration.

15. The method of claim 13, wherein said mobilization means is Mozibil administration.

16. The method of claim 13, wherein said mobilization means is FLR-3L administration.

17. The method of claim 9, wherein said type 2 cytokine polarization is associated with reduction in ability to produce interferon gamma after mitogenic stimulation.

18. The method of claim 17, wherein said mitogenic stimulation is treatment with phytohemagluttinin at a concentration of 2 micrograms per ml.

19. The method of claim 9, wherein said type 2 cytokine polarization is associated with enhanced IL-4 production after mitogenic stimulation.

20. The method of claim 19, wherein said mitogenic stimulation is treatment with phytohemagluttinin at a concentration of 2 micrograms per ml.
